(19) United States
(12) Patent Application Publication
(10) Pub. No.: US 2011/0118757 A1
(43) Pub. Date: May 19, 2011

(54) SURGICAL END EFFECTOR APPARATUS AND METHOD

(75) Inventor: Javin C. Pierce, Stowe, VT (US)

(73) Assignee: PIERCE INSTRUMENTS, INC., Stowe, VT (US)

(21) Appl. No.: 12/293,999

(22) PCT Filed: Mar. 22, 2007
(86) PCT No.: PCT/US2007/007216

§ 371 Date: Nov. 5, 2010

Related U.S. Application Data
(60) Provisional application No. 60/784,909, filed on Mar. 22, 2006.

(51) Int. Cl. A61B 17/04

(52) U.S. Cl. 606/144

(57) ABSTRACT

A surgical end effector apparatus and method places suture materials in tissue inside the body. The surgical end effector comprises a hollow sharp elongated cannula that conveys an internal flexible shaft suture conveyance that is advanced after the assembly is inserted in the tissue. When advanced beyond the inserter, the tip of the internal shaft can move laterally by spring rebound force and/or a suture tension can also be used to induce lateral displacement of the tip to form a taught bowstring array, to facilitate suture retrieval by positioning the suture at a right angle to the inserter cannula, and by further rotation of the of the taught bowstring array. The tip of the shaft can also be sharp to enable lateral puncture of tissue. An alternative embodiment comprises a suture retriever arm.

### RELATED APPLICATIONS

This application claims priority to U.S. provisional application Ser. No. 60/784,909, filed Mar. 22, 2006. The entire contents of this reference is incorporated by reference herein.

### FIELD

The embodiments disclosed herein relate to a medical device for suture placement, tissue repair, and surgical access, and more particularly to a surgical end effector where one element is tensioned relative to another to induce movement, and methods for using a surgical end effector for suture placement, tissue repair, and surgical access.

### BACKGROUND

Numerous and various types of suture placing devices are designed to facilitate the repair of tissue by way of passing surgical sutures, but these are limited in their ability to pass sutures laterally after making a minimal axial puncture of the tissue.

Tubular, wire rope, and solid, spring-action, medical devices are commonly used for catheter guidewire, endoscope control cable, and catheter shaft applications, but these are not used to convey a suture through tissue, do not comprise a suture receiving means.

The prior art suturing placing and suturing procedures present many problems to the medical professional and the patient. A curving suture end effector commercially available from Arthrex, Inc. in Naples, Fla. comprises a flexible pre-bent spring-form nitinol wire with a smaller wire loop suture receiving means on its tip for the purpose of introducing a suture through an elongated tube or blunt cannula through a hole in bone. End effect movement occurs due to the spring rebound effect of the pre-bent element seeking its equilibrium upon being released from the introducer tube.

Prior art suture placing and suturing devices for use by medical professionals are known in the art. U.S. Pat. No. 6,991,636 entitled “Nitinol Loop Suture Passer” discloses a nitinol flexible alloy wire array comprising a clamping element to form a suture receiving means, that similarly employs only release-induced spring action to effect movement of the tip.

U.S. Pat. No. 5,972,005 entitled “Wound Closure Assembly and Method of Use” discloses a suturing device employing pre-curved needles that are ejected out of the assembly and assume a pre-curved shape and in the process curve back to penetrate tissue prior to reentering the assembly.

U.S. Pat. No. 6,730,097 entitled “Surgical Snare with Steering Tether and Method of Using Same” discloses a surgical instrument having a flexible cable slidably positioned in a passageway. A snare loop is attached to the cable. A steering tether is attached to the loop so that application of a tensile force to the tether causes the loop to deform in a predetermined manner. The loop can also be a suturing material.

U.S. Pat. No. 5,336,231 entitled “Parallel Channel Fixation, Repair and Ligation Suture Device” discloses a device having a central passageway with parallel channels on opposite sides. A suture extends through the central passageway and has a loop.

U.S. Pat. No. 6,554,845 entitled “Suturing Apparatus and Method” discloses a suturing apparatus including a first member and a needle member, wherein the first member and the needle member are disposed to permit selective relative movement therebetween.

The prior art spring action devices can become caught in or against tissue structures, especially in the close confines of the posterior knee joint, and lack sufficient torsional strength to impart rotation around obstructions. The prior art devices do not incorporate a flexible element that comprises a central suture receiving lumen, nor do these use a bimorph tension-compression element in the form of suture or another part, to impart movement both lateral and rotational movement for the purpose of passing a suture or intermediary suture shuttle device.

Thus, there is a need in the art for an apparatus and method for a surgical end effector that is safe, reliable, user friendly, and effective.

### SUMMARY

An apparatus and method for a surgical end effector is disclosed herein. According to one embodiment of the invention, a surgical end effector is provided comprising an elongated cannula, a shaft assembly, a suture receiver, and a suture apprehensive retriever. The elongated cannula has a sharp distal end and a proximal end engaging an operator interface. The shaft assembly is slidably disposed within the cannula. The shaft assembly includes a tip that moves from a first laterally confined position to a second position when the tip extends beyond the distal end of the cannula. The suture receiver is located on the tip and is capable of engaging a suture. The suture apprehension retriever is designed to releasably capture a laterally disposed suture to tie or otherwise connect with another portion of the same suture.

The surgical end effector can have various configurations. For example, the shaft assembly may be pre-bent. The elongated cannula may have a curved distal portion. The surgical end effector may include a tension element to laterally displace the tip. The tension element may be a suture in some embodiments, but may also be any device capable of applying tension including, but not limited to, fiber, string, rope, wire, wire rope, cable, chain, and/or mechanical linkage. The elongated cannula may be hollow. The shaft assembly may be solid or hollow.

The shaft assembly may include one or more lumens and one or more holes. In some embodiments, a suture is releasably conveyed through at least one of the one or more lumens and passes out of the shaft assembly through at least one of the one or more holes. In other embodiments, the shaft assembly comprises two lumens and two holes, wherein a suture is releasably conveyed through a first lumen, passes out of the shaft assembly through a first hole, passes into the shaft assembly through a second hole and is releasably conveyed through a second lumen.

The shaft assembly may include a flexible wire and/or a spring form. In other embodiments, the operator interface applies tension to a suture. The suture receiver may be a hole in the tip. The suture apprehension retriever may include an outer sheath having a lumen and a distal end and an inner retriever having a distal retrieval hook. The inner retriever may be slidably positioned in the lumen of the outer sheath.

Also provided is a surgical end effector including an elongated shaft assembly having distal end and a proximal end. A flexible portion of the shaft assembly is located toward the distal end of the shaft assembly. A tip is located at the distal end of the shaft assembly. The tip is movable from a first position to a second flexed position. A suture receiver is located adjacent to the tip.

The surgical end effector can have various configurations. The surgical end effector may include a suture engaging the suture receiver. The surgical end effector may include a suture apprehension retriever to releasably capture a laterally disposed suture to tie with another portion of the same suture. The surgical end effector may also include an elongated tube having a sharp distal end and a proximal end engaging an operator interface. The elongated shaft assembly may be slidably and rotationally movable within the elongated tube. In some embodiments, the surgical end effector includes a tension element adjacent to the tip. The tip may be a cutting'or puncturing tip such as a knife or a trocar. Additionally or alternatively, the tip may be detachable. The suture receiver may be an eyelet. In other embodiments, the shaft assembly comprises a plurality of legs and tip movement is achieved by pushing a first subset of the legs while pulling a second subset of the legs.

Also provided is a method for surgical suture placement. The method includes penetrating tissue with an elongated substantially straight sharp instrument, advancing an elongated shaft within the sharp instrument, and applying tension to a suture tension element or other tensioning means to move the tip and suture holder to a lateral position. The shaft includes a tip extending from a flexible portion and a suture engaged to a suture holder located on the tip of the shaft. The tip is movable in at least one lateral direction.

The method can have several variations. For example, the method may include rotating the suture holder and suture tension element. The method may also include retrieving the suture with a releasable slidable retriever.

Another aspect of the invention is a surgical end effector comprising a flexible shaft, a suture receiver, and an internal stiffening element. The flexible shaft includes a sharp distal tip and an internal lumen. The suture receiver is located toward the distal tip. The internal stiffening element is received in the lumen and enables the flexible shaft to be rigid during tissue penetration. The stiffening element may be withdrawn from the flexible shaft to effect lateral movement of the tip while managing suture tension.

The surgical end effector can have several embodiments. For example, the internal stiffening element can be a wire. A suture can form a taught bowstring array. The distal portion of the flexible shaft can be flexible. A proximal end of the internal stiffening element may be partially withdrawn from the flexible shaft while a distal end of the internal stiffening element remains in the flexible shaft.

Another aspect of the invention is a surgical end effector for fluid injection including a shaft assembly. The shaft assembly includes a shaft, a tip, a fitting interface, and a lumen. The tip is located at the distal end of the shaft and is movable from a first position to a second flexed position. The lumen is in communication with the fitting interface. Fluid can be received through the fitting interface and the lumen before exiting the lumen.

The surgical end effector can have several embodiments. For example, the tip may be a sharp tip. The lumen may be internal or external to the shaft. The surgical end effector may also include an elongated tube having a distal end and a proximal end. The distal end of the elongated tube may be sharp or blunt. The shaft assembly may also include a tension element adjacent to the tip.

The surgical end effector may receive a variety of fluids. For example, the fluid may be a flowable tissue adhesive. The fluid may include one or more of the following: blood constituents, cells, drugs, growth factors, nutrients, growth scaffolds, bioactive agents, insulin, gene therapy agents, plasmid DNA, naked plasmid DNA, and elements.

Another aspect of the invention is a surgical end effector including an elongated cannula, a fluid injector, a shaft, a suture receiver, and a suture apprehension retriever. The elongated cannula has a sharp distal end and a proximal end which engages an operator interface. The fluid injector is for injecting fluids through the elongated cannula. The shaft assembly is slidably disposed within the cannula. The shaft assembly comprises a tip that moves from a first laterally confined position to a second position when the tip extends beyond the distal end of the cannula. The suture receiver is located on the tip for engaging a suture. The suture apprehension retriever is designed to releasably capture a laterally disposed suture to tie or otherwise connect with another portion of the same suture.

The fluid injector may inject a variety of fluids through the elongated cannula. For example, the fluid may be a flowable tissue adhesive. The fluid may include one or more of the following: blood constituents, cells, drugs, growth factors, nutrients, growth scaffolds, bioactive agents, insulin, gene therapy agents, plasmid DNA, naked plasmid DNA, and elements.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 is a perspective view of a surgical end effector of the presently disclosed embodiments with a solid flexible shaft, curved-tip sharp cannula, and ergonomic handle.

FIG. 2 is a longitudinal cross sectional view of a surgical end effector of the presently disclosed embodiments with a solid flexible shaft, curved-tip sharp cannula, outer sheath, suture retrieval means and ergonomic handle.

FIG. 3 is a perspective exploded view of a surgical end effector of the presently disclosed embodiments with a solid flexible shaft, curved-tip sharp cannula, and ergonomic handle.

FIG. 4 is longitudinal cross sectional view of a surgical end effector of the presently disclosed embodiments with a solid flexible shaft, curved-tip sharp cannula, and ergonomic handle, after positioning in tissue, and shows an initial view of a sequence depicting a method of use.

FIG. 5 is a view showing a solid flexible shaft of a surgical end effector being advanced distally and the flexible shaft suture receiving means displacing laterally to the curved-tip sharp cannula.

FIG. 6 is a view showing a solid flexible shaft of a surgical end effector being further advanced distally and the flexible shaft suture receiving means displacing laterally to the curved-tip sharp cannula.

FIG. 7 is a view showing a solid flexible shaft of a surgical end effector being advanced distally and the flexible shaft suture receiving means displacing laterally to the curved tip sharp cannula to form a tensioned bowstring array for suture apprehension.

FIG. 8 is a view showing a solid flexible shaft of a surgical end effector immobile in the curved-tip sharp cannula and suture tension being applied to tighten and reduce the arc of the bowstring array, reversing lateral displacement toward the introducer, and disposing the bowstring array more proximally toward the operator and into the visual field to facilitate suture apprehension.

FIG. 8A is a view showing the rotational positioning of the bowstring array.

FIG. 9 is a view showing an alternate method of use in the sequence shown in FIG. 4 and FIG. 5 where after the situation of FIG. 5, the operator restrains further pay-though of suture but advances the flexible shaft.

FIG. 10 is a view showing the step following that of FIG. 9 of advancement of the solid flexible shaft with suture restraint to obtain a tethered lateral displacement of the solid flexible shaft tip along an arc.

FIG. 11 is a view showing the step following that of FIG. 10 depicting the solid flexible shaft being advanced distally and the solid flexible shaft suture receiving means and tethered bowstring array reversing lateral displacement to the introducer, and disposing the bowstring array more proximally and into the visual field to facilitate suture apprehension, along an arc. FIG. 11 also depicts a typical disposition of the elements and tissue after the step depicted in FIG. 8 where suture tension was applied to effect the same or similar resulting disposition of elements.

FIG. 12 is a view showing the step following that of FIG. 11 where a suture retrieval device is used to apprehend the suture.

FIG. 13 is a view showing the step following that of FIG. 12 where a suture retrieval device apprehends the suture and draws the suture around the tissue to be repaired.

FIG. 14 is a view showing the step following that of FIG. 13 where the leg of suture passing through the sharp cannula has been released from the handle or the hand of the operator and the end of the suture can pay out through lumen of the sharp cannula.

FIG. 15 is a view showing the step following that of FIG. 14 where the solid flexible shaft has been withdrawn into the sharp cannula and the free end of suture has been withdrawn through the outer sleeve by the suture retrieval means.

FIG. 16 is a view showing the step following that of FIG. 15 where the sharp cannula and flexible shaft have been removed from the tissue leaving the suture ready to be tied to itself forming a repair closing a tissue tear.

FIG. 17 is a perspective view of a surgical end effector with a hollow-lumen flexible shaft and straight-tipped sharp cannula of the presently disclosed embodiments.

FIG. 18 is a longitudinal cross sectional view of a surgical end effector with a hollow-lumen flexible shaft and straight-tipped sharp cannula of the presently disclosed embodiments.

FIG. 19 is a perspective exploded view of a surgical end effector with a hollow-lumen flexible shaft and curved-tipped sharp cannula of the presently disclosed embodiments.

FIG. 20 is a longitudinal view of an alternative flexible shaft tip design where by the distal end of the flexible shaft is tipped with a tissue cutting tip with a transverse hole suture receiving means.

FIG. 20A is a cross sectional view of the suture retrieval means apprehending the suture leg and the detachable tip.

FIG. 21 is a longitudinal view of a flexible shaft with a flexible spring form distal portion.

FIG. 22 is a longitudinal view of an embodiment of a flexible shaft element whereby the suture is attached to the flexible shaft by means of a wire loop eyelet suture receiving means.

FIG. 23 and FIG. 24 are longitudinal cross section views depicting a sequence of steps whereby an internal stiffening element enables the flexible shaft to be rigid during tissue penetration whereby removal of the stiffening element effects lateral movement of the tip.

FIG. 25 is a longitudinal cross section view that depicts a bimorph embodiment of a flexible shaft whereby the flexible shaft comprises a wire loop suture receiving eyelet with a collar near the tip and two wire legs.

FIG. 26 is a perspective view of a flexible shaft whereby three or more push-pull elements may be used to impart motion in additional lateral axes or to impart more force by gang action.

FIG. 27 and FIG. 28 are longitudinal cross section views of an embodiment of a hollow flexible shaft comprising a two-opening sharp tip head whereby the two openings communicate with the lumen of the shaft. FIG. 27 depicts one option of using this embodiment where one leg of the suture is run through the lumen and the other leg of the suture is external to the lumen. FIG. 28 depicts a second option of using this embodiment whereby both legs of the suture are run outside the lumen of the flexible shaft.

FIG. 29 is a longitudinal cross section view of an embodiment of a flexible shaft having two lumens whereby a suture may be run through one lumen of the flexible shaft, exit the shaft through a hole and reenter the shaft through another hole into the second lumen.

FIG. 30 is a longitudinal cross section view of the surgical end effector with liquid injection means being used to inject a flowable material into a selected tissue region, in this case a portion of the prostate gland to be treated by the flowable material.

FIGS. 31A, 31B, 31C, and 31D are longitudinal section views of a sheathed/slidable suture retrieval means whereby the suture retrieval means is capable of selectively apprehending and releasing a suture.

FIGS. 32, 33, and 34 depict a flowable tissue adhesive being applied to a tear in a meniscus prior to retraction of the device. FIG. 32 and FIG. 33 depict two separate methods of applying adhesive.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

A surgical end effector and method of surgery operates inside the body where space, access, and visualization are limited. The surgical end effector passes the suture through and/or around structures to repair tissue with the suture, although other uses are anticipated. The surgical end effector can be used to repair knee meniscus tissue by means of arthroscopic surgery. The surgical end effector can be used to pass sutures through tissue by means of arthroscopic surgery to repair tears of the shoulder rotator cuff, and numerous other endoscopic suturing applications are within the scope of the present invention.

In one embodiment, the surgical end effector comprises an elongated cannula with a sharp distal tip suitable for penetration of soft tissue, the proximal end having a handle or operator interface. The surgical end effector also includes an internally disposed, slidable pre-bent or controllably bendable flexible shaft comprising a suture receiving means in or near its distal tip that is fully retracted and held straight during positioning or tissue penetration. The flexible shaft comprises a suture receiving means adjacent to a sharp distal tip whereby after the flexible shaft is advanced distally to emerge from the sharp cannula it can track laterally from the cannula penetrating tissue in a secondary direction until the tip is clear of tissue.

Tension is applied to the suture and the flexible shaft tip by the operator selectively limiting the distal travel of the suture to an amount less than the distal travel of the flexible shaft, or by retracting the suture to pull the flexible distal tip in a proximal direction to further bend the flexible shaft and induce it to further displace toward the operator and/or the hole in the tissue formed by the shaft of the sharp cannula. The direct or indirect operator-induced suture tension results in a taught bowstring effect whereby lateral displacement of the flexible tip array occurs, and can be optionally combined with operator induced rotation of the flexible element-bowstring array via an operator interface, thereby facilitating operator suture positioning for capture and subsequent fastening of suture to form a secure repair.

The surgical end effector has a bimorph functionality whereby one element is tensioned relative to the other to induce movement. The term “bimorph” is used broadly to encompass the combination of tension and compression forces. These forces could be generated by mechanical means or could be generated by bimorph thermal or electrical activation as known in the art. Because the suture is laterally confined by tissue or the sharp cannula near the distal end of the surgical end effector, a pulley point is formed that creates end effector movement in a manner similar to an animal tendon flexing a joint. In an embodiment, a flexible means such as a sleeve, dovetail, or adhesive holds the compressive and tension elements close to each other. This embodiment has the tension and compression elements affixed to each other at a point and the movement of the bimorph causes the end effector to curl without comprising a taught bowstring array. This embodiment is particularly useful for applications where a suture is not used.

An embodiment of the surgical end effector has a unique design to act as a suture placer. The end effector has an internally disposed hollow bore in the flexible shaft, comprising multiple metallic wire filars, that conveys suture through the hollow bore in the length of the flexible shaft. Another embodiment comprises a solid flexible shaft, a sharp tip, and suture affixed near said tip. The surgical end effector enables surgeons to pass the suture through and/or around obstructions. The operator can selectively advance the flexible element past the confines of the insertion cannula to enable the flexible spring-form element to articulate under operator control via the suture or tension element control. The surgical end effector forms a taught bowstring array with the suture or a compliant tension element in a lateral disposition from the insertion cannula so that it can be retrieved by a common hook or grasper instrument and the suture can be drawn over or around the tissue to be repaired. The surgical end effector comprises the hook or grasper suture retrieval means that is integrated into the shaft of the inserter cannula forming a bifurcation, for the purpose of guiding it around the target tissue to coact with the taught bowstring to retrieve the suture. The surgical end effector conveys the suture or the bowstring tension element into the visual field of the surgeon to assist suture withdrawal and the successive step of suture tie off of the repair. The integrated suture embodiment does not necessarily require visualization to convey the suture to the retriever.

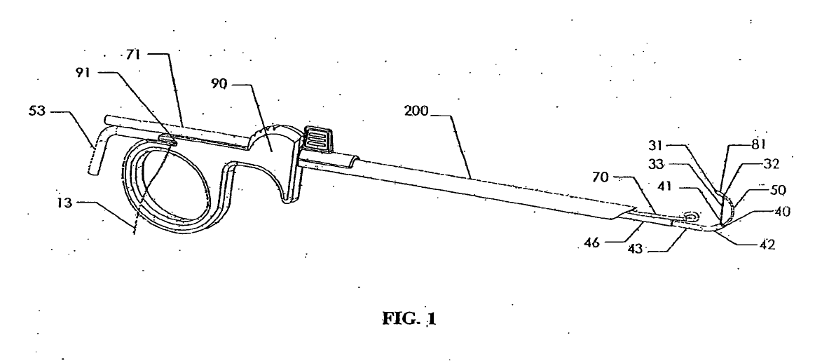

FIG. 1 is a perspective view of the surgical end effector including a solid flexible shaft 50, the shaft 50 comprising a swaged tip section 81 with a sharp tip 31, a suture receiving means 32, a handle for solid flexible shaft 53, a suture 10 (not fully shown) comprising an external leg 11 running through the sharp cannula 43 by means of the sharp cannula lumen 41. The solid flexible shaft 50 is housed within sharp cannula lumen 41, and sharp cannula 43 and the flexible shaft 50 can selectively extend beyond a sharp cannula tip 40 to convey suture 10. The surgical end effector further comprises a suture retrieval means 70 with a suture retrieval means handle 71. The flex shaft 50 and suture retrieval means 70 pass through an ergonomic handle 90. The surgical end effector further comprises an outer sleeve 200 that covers portions of the ergonomic handle 90, sharp cannula 43, and suture retrieval means 70. The suture retrieval means 70 can selectively extend beyond the outer sleeve 200 to apprehend the external leg 11 of the suture 10.

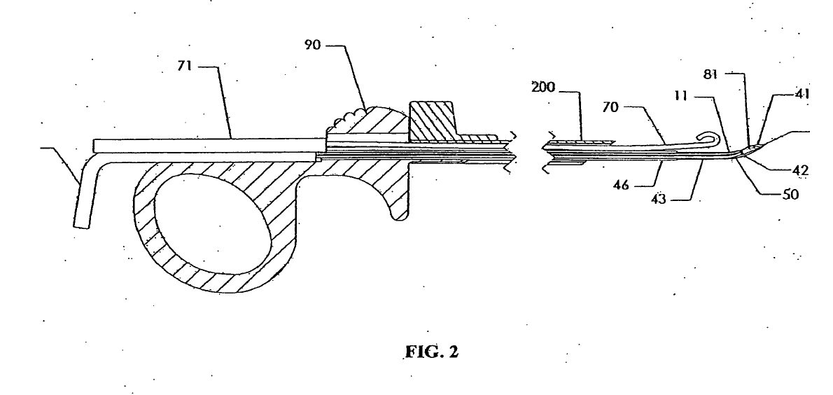

FIG. 2 is a longitudinal cross sectional view of the surgical end effector showing the solid flexible shaft 50, and the swaged tip section 81 with sharp tip 31 fully retracted within the sharp cannula lumen 41, for the purpose of insertion into tissue across the defect to be closed.

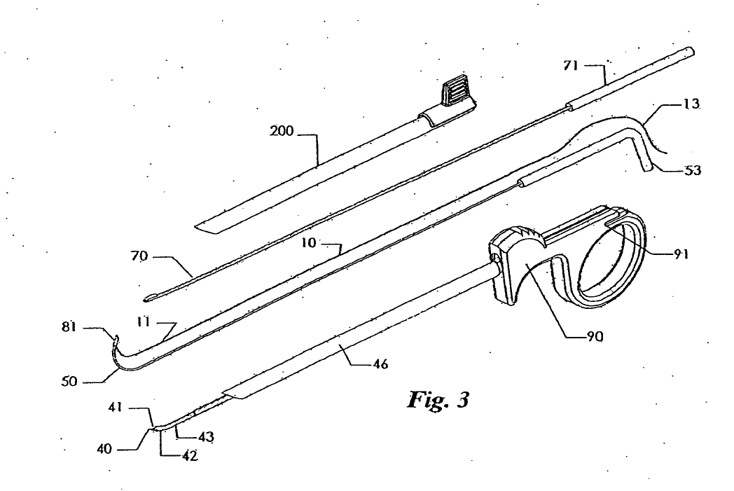

FIG. 3 is a perspective assembly view of the surgical end effector. A single strand of suture 10 is attached to the flexible shaft 50 by a suture receiving means 32 and is used as a tensioning means for the flexible end effector. The suture 10 is external to the solid flexible shaft 50. The solid flexible shaft 50 and suture 10 pass through the sharp cannula 43 by means of the sharp cannula lumen 41. The free end 13 of the suture 10 may be wedged in place by pulling the suture end 13 into a suture cleat 91. Other embodiments comprise a releasable suture locking means such as notches, catches, and similar structures known to those skilled in the art.

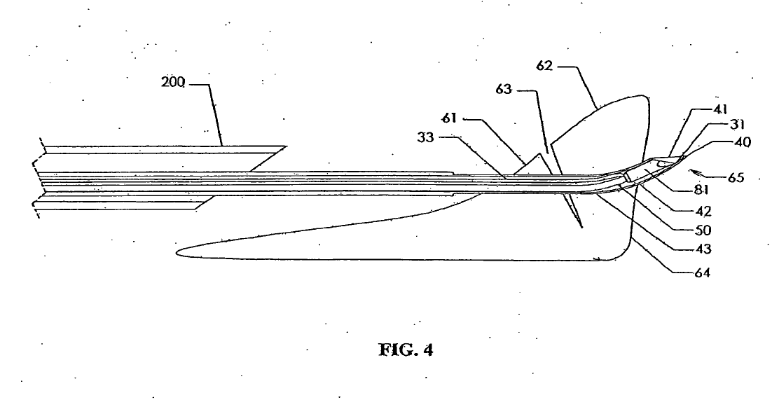

FIG. 4 is the first in a series of figures illustrating the sequence and method of use for the surgical end effector of the presently disclosed embodiments. FIG. 4 shows the surgical end effector after the sharp cannula tip 40 has penetrated a central portion of meniscus 61, a crossed meniscus tissue tear 63, penetrated a posterior meniscus 62, 64, and the distal end of the sharp cannula lumen 41, has emerged into the space 65 behind the meniscus 61, 62, 64. The sharp cannula 43 has a bend 42 that serves to guide the swaged tip 81 of the solid flexible shaft 50 in a lateral direction. The displacement of the surgical end effector into tissue in FIG. 4 is accomplished by user manipulation of the sharp cannula 43 by the handle 53 for solid flexible shaft 50 (refer to FIG. 1).

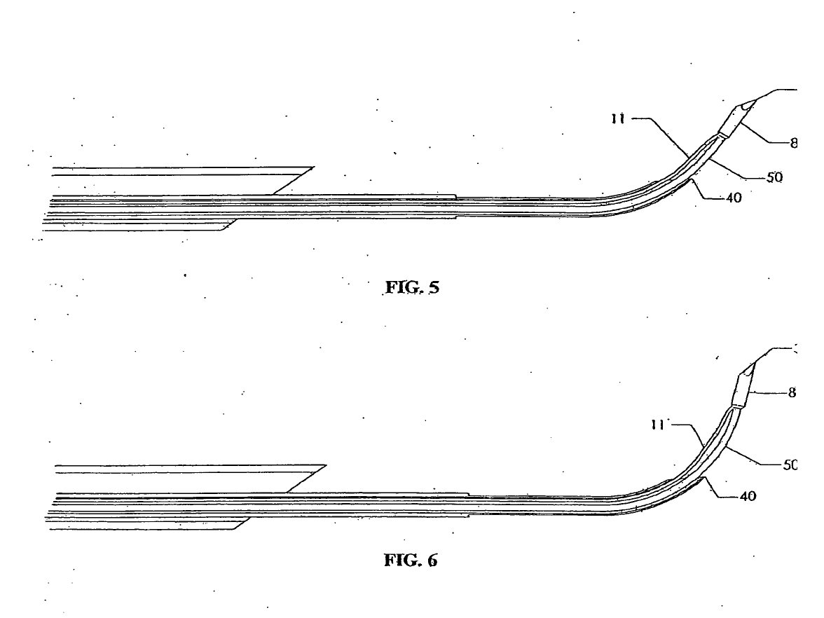

FIG. 5 depicts the flexible shaft 50 after it has been advanced beyond the sharp cannula tip 40 by operator movement of the solid flexible shaft handle 53 (refer to FIG. 1). The tip of the flexible shaft 50 begins to displace laterally as it advances to form a bowstring array with the suture 10. In an embodiment, the flexible shaft 50 is pre-bent into a spring form that will rebound from a substantially straight disposition (shown in FIG. 2), to a curved position when advanced from the sharp cannula lumen 41 as in FIG. 5. In the sequence of FIG. 5 to FIG. 6, as the flexible shaft 50 is advanced, the suture 11 is pulled outward through the sharp cannula lumen 41. The flexible shaft sharp tip 31 is capable of piercing tissue that may otherwise restrict its movement, thereby enabling the surgical end effector to pierce tissue at a second, nearly 90 degree angle, to the first path of tissue penetration established by sharp cannula 43. This ability is useful in surgery where the sharp cannula 43 emerges from the posterior meniscus 62, 64, beneath a layer of soft tissue such as a capsule.

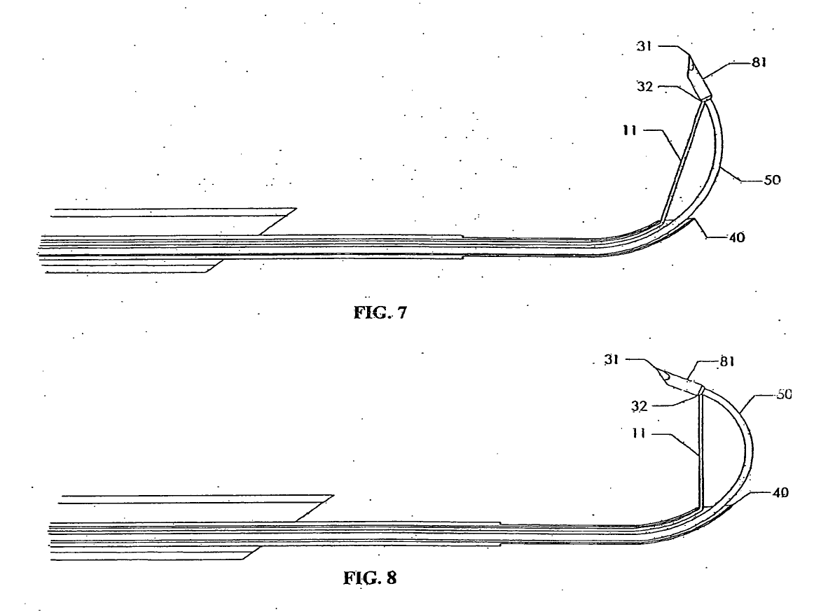

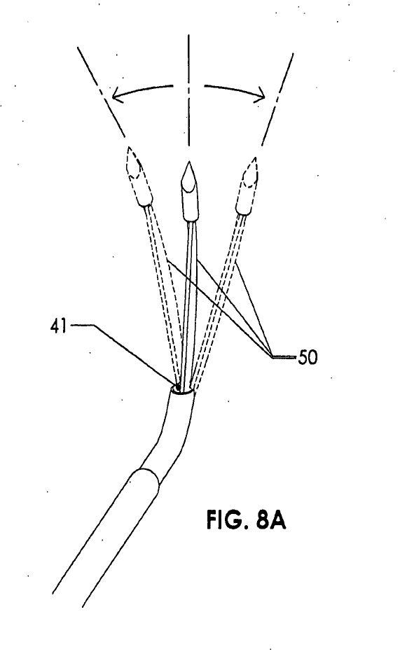

In FIG. 6, FIG. 7 and FIG. 8, the surgical end effector is depicted with the solid flexible shaft 50 fully extended. In FIG. 7 tension is applied by the operator to the bowstring array via the suture 11 in order to convey the flexible shaft sharp tip 31, and the flexible shaft suture receiving means 32, in a proximal direction to facilitate capture of the suture 11 that comprises the bowstring array. The surgical end effector enables the movement of the tip 31, distally as shown in FIG. 4, distally and outward-laterally as shown in FIG. 5 and FIG. 6, outward-laterally and proximally as shown in FIG. 7, and proximally and inward-laterally as shown in FIG. 8.

FIG. 8A depicts rotational positioning of the bowstring array. The surgical end effector also allows the operator to selectively rotate the entire flexible shaft 50 by means of rotating the handle 53 for solid flexible shaft 50 (refer to FIG. 1) thereby rotationally positioning the taught bowstring array within the limits created by the angular shape and disposition of sharp cannula lumen 41 (refer to FIG. 4).

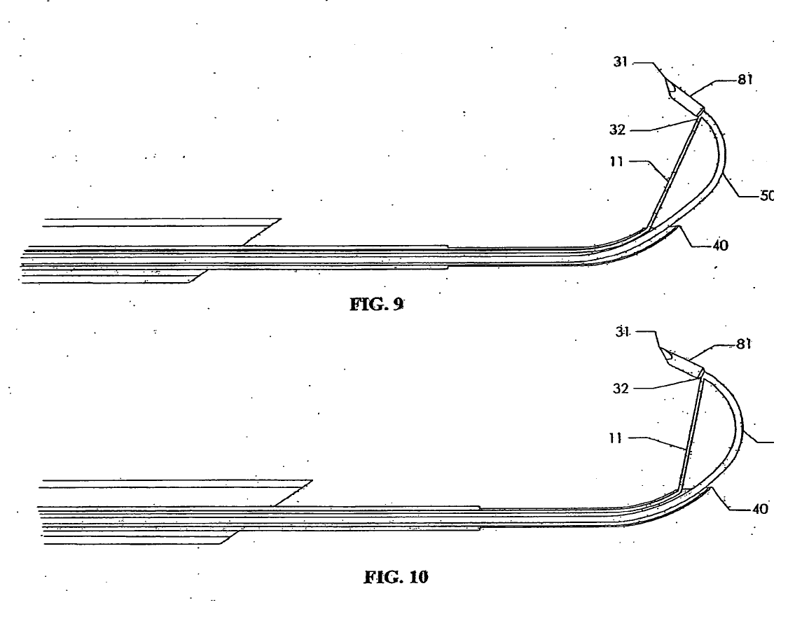

FIG. 9 depicts an alternate method in the sequence shown in FIG. 4 and FIG. 5 where after the situation of FIG. 5 the operator restrains further pay-though of the suture 11 (though the sharp cannula lumen 41), but advances the flexible shaft 50. The restraint of suture 11 can be achieved by means of a cleat 91, groove, screw, or other releasable suture holding means incorporated into, or affixed to the handle for solid flexible shaft 53.

FIG. 10 depicts the step following that of FIG. 9 of advancement of the flexible shaft 50, with suture restraint to obtain a tethered lateral displacement of the flexible shaft tip 31 along an arc.

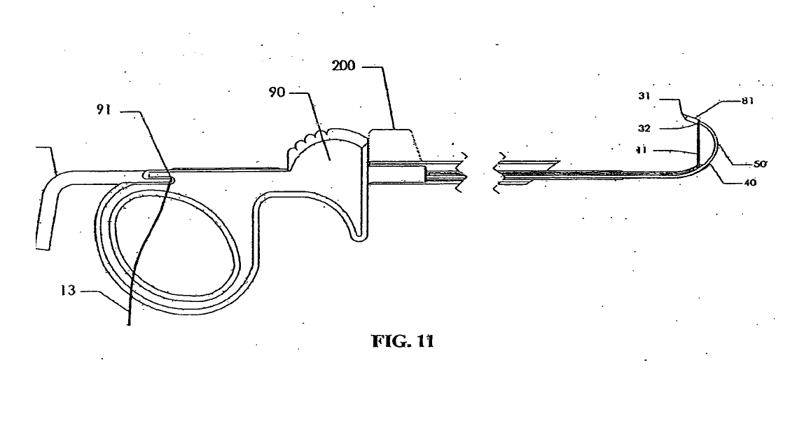

FIG. 11 depicts the step following that of FIG. 10 depicting the flexible shaft 50, being advanced distally and the flexible shaft suture receiving means 32, and tethered bowstring array reversing lateral displacement inwardly toward sharp cannula 43, and disposing the bowstring array more proximally and into the visual field to facilitate suture apprehension, along an arc. FIG. 11 also depicts a typical disposition of surgical end effector after the step depicted in FIG. 8 where suture tension was applied to effect the same or similar resulting disposition of elements.

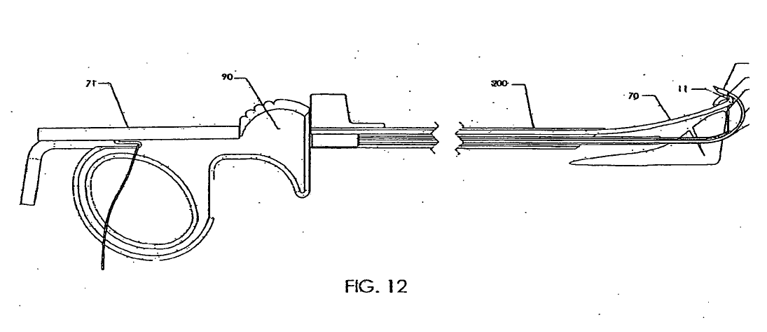

FIG. 12 depicts the step following that of FIG. 11 where a suture retrieval device 70 is used to apprehend the suture end 11. The suture retrieval device 70 is advanced through the ergonomic handle 90 by means of manipulating the suture retrieval device handle 71. The suture retrieval device 70 travels through and extends beyond the outer sheath 200.

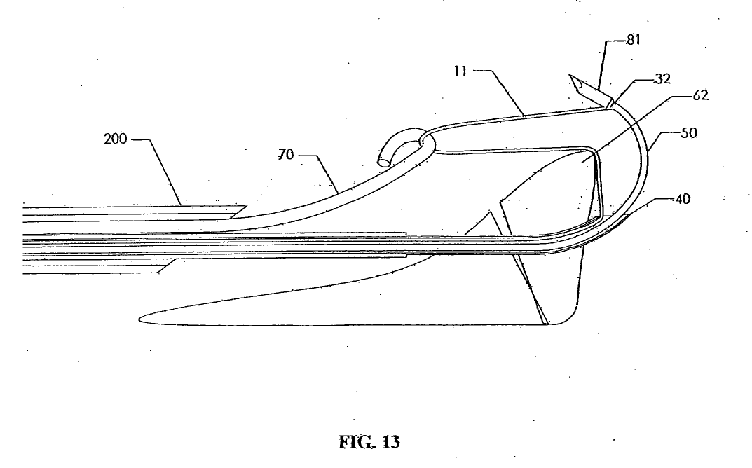

FIG. 13 depicts the step following that of FIG. 12 where a suture retrieval device 70 apprehends and captures the suture end 11, and draws the suture end 11 around the posterior meniscus tissue 62 to be repaired. The suture retrieval device 70 moves the suture end 11 to form a repair closing a tissue tear 63.

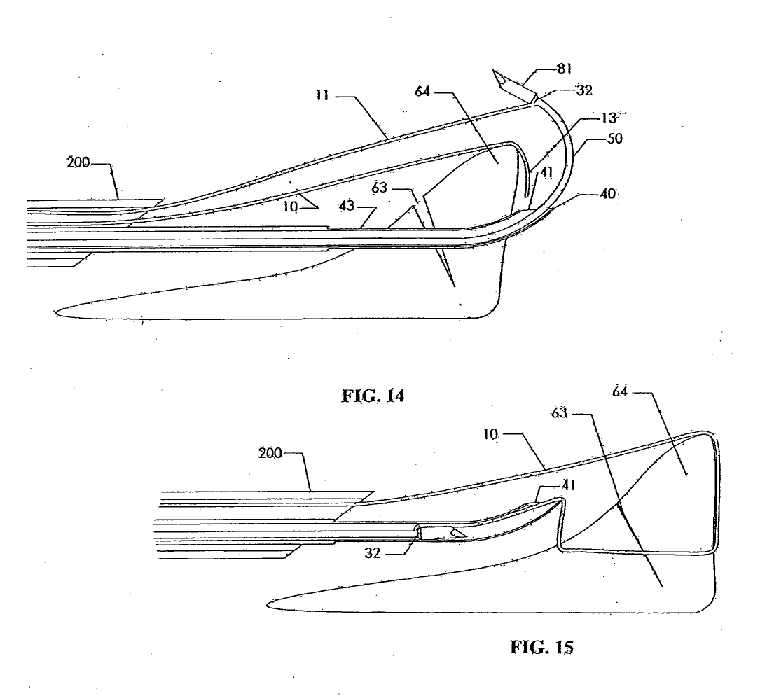

FIG. 14 is a view showing the step following that of FIG. 13 where the suture 10 has been released from the handle 90 or the operator's hand and the free end 13 of the suture 10 can pay out through the lumen 41 of the sharp cannula 43.

FIG. 15 is a view showing the step following that of FIG. 14 where the flexible shaft 50 has been withdrawn into the sharp cannula lumen 41, pulling the suture receiving means 32 and suture 10 into the sharp cannula 43. The free end 13 of the suture 10 has been pulled through the lumen of the outer sheath 200.

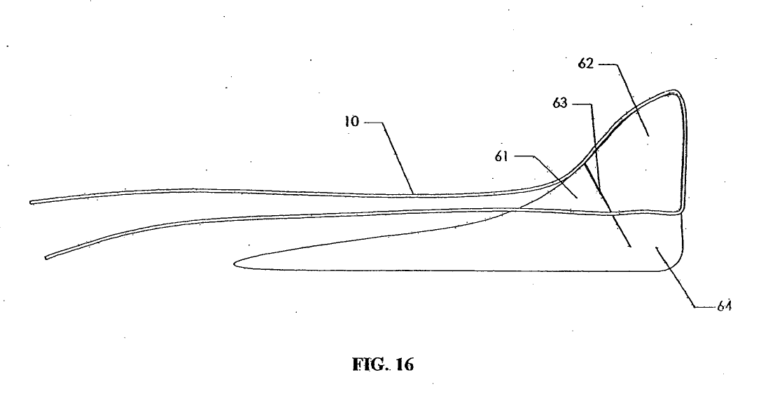

FIG. 16 is a view showing the step following that of FIG. 15 where the sharp cannula 43 has been removed from the tissue 61, 62, 64 and the suture 10 has been released from the suture receiving means 70, leaving the suture 10 free of the instrument so that the suture 10 may be tied to itself forming a repair closing tissue tear 63. The suture 10 forming the repair is no longer engaged to the surgical end effector.

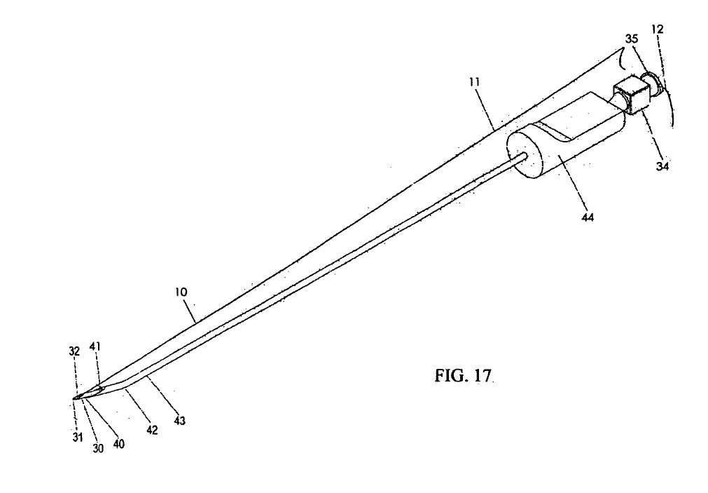

FIG. 17 is a perspective view of the surgical end effector including an elongated hollow flexible shaft 30, the shaft 30 comprising a sharp tip 31, a suture receiving means 32, a flexible shaft handle 34, a flexible shaft liquid injection means 35, a suture 10 comprising an external leg 11, and an internal suture leg 12 running through the hollow flexible shaft 30 by means of a flexible shaft lumen 36. The flexible shaft 30 is housed within sharp cannula lumen 41, sharp cannula 43, and handle 44. The flexible shaft 30 can selectively extend beyond a sharp cannula tip 40 to convey suture 10.

A surgical end effector having a solid flexible shaft 50 may be configured to operate in the same or similar manner to an end effector having a hollow flexible shaft 30 as described herein through the introduction of one or more external lumens to the solid flexible shaft 50. The lumens could be attached to the solid flexible shaft by any means known to those of skill in the art including adhesives, epoxies, welding, tack welding, and fasteners. In some embodiments, one or more of the external lumens may have a sharp tip to pierce tissue for fluid injection.

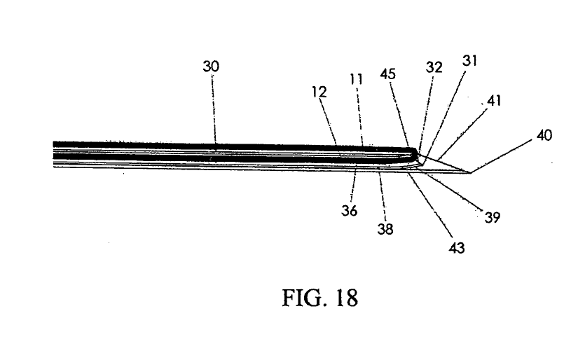

FIG. 18 is a longitudinal cross sectional view of the surgical end effector showing the flexible shaft 30, and the flexible shaft sharp tip 31 fully retracted within the sharp cannula lumen 41, for the purpose of insertion into tissue across the defect to be closed. FIG. 18 depicts an embodiment of the invention where the sharp cannula 43 is straight rather than curved.

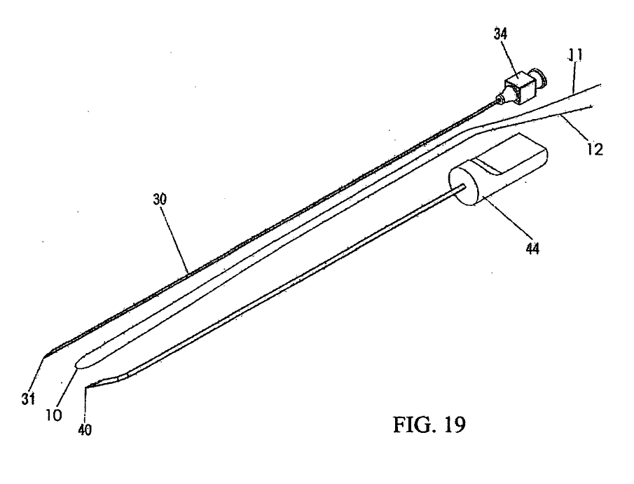

FIG. 19 is a perspective exploded view of the surgical end effector. A single strand of suture 10 is used as a tensioning means for the flexible end effector, and the suture 10 is used as an elongated loop having a leg 11, that is external to the flexible shaft 30, and flexible shaft handle 34. The internal leg 12 of suture 10 is placed within the flexible shaft 30 through an opening near the tip 31 of said shaft 30 and leg 12 runs through the flexible shaft lumen 36 (see FIG. 18). Internal leg of suture 12 can be locked to the handle 34 by a coacting interference fitting (not shown) that will render the suture leg 12 immobile to facilitate operator tensioning of the external leg 11. Other embodiments comprise a releasable suture locking means such as cleats and similar structures known to those skilled in the art.

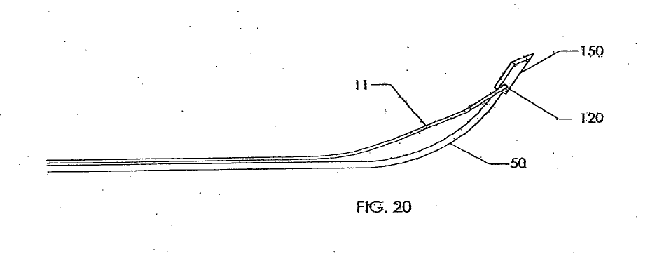

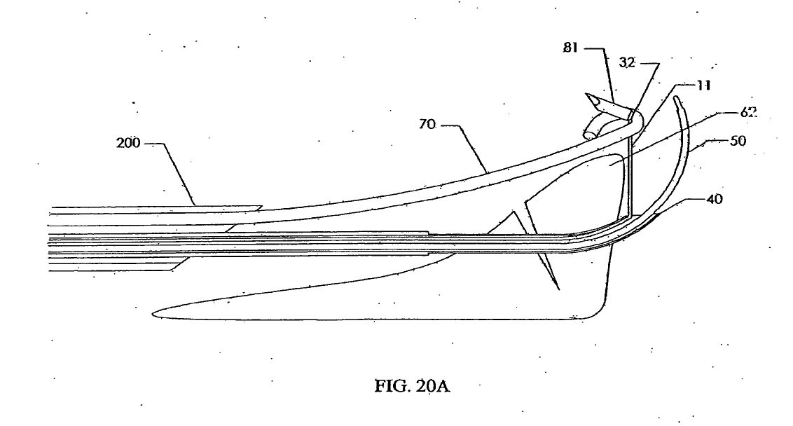

FIG. 20 depicts an alternative tip design whereby the distal end of the solid flexible shaft 50 is tipped with a tissue cutting tip 150 with a transverse hole 120 suture receiving means. The cutting tip may have several embodiment and including a knife blade and/or a trocar. A cutting tip 150 may also be used on other configurations of flexible shafts 30, 50 described elsewhere in this document.

FIG. 20A depicts another alternative tip design whereby the tip section 81 is detachable from the flexible shaft 30, 50 but it remains affixed to suture 10. A detachable tip section 81 may be advantageous in certain situations because the tip section 81 may be released before retraction of the flexible shaft 30, 50. This may facilitate suture passing by reducing friction, and possible damage to suture 10. Moreover the detachable tip section 81 feature could reduce the possibility of prematurely detaching the suture 10 from the passer. In some embodiments, the tip section 81 may be engineered to sheer off if a defined resistance is met.

As depicted in FIG. 20A, the tip section 81 may be detached after the bow string array formed by suture leg 11 is captured by the suture retrieval means 70. In some embodiments, the tip section 81 remains connected to the suture leg 11 through the attached suture receiving means 32. The tip section 81 is then pulled back into the sheath 200 by the suture retrieval means 70. The shaft 50 then has a lower cross section when retracted through the tissue.

The shaft 50 depicted in FIG. 20 may be deployed in a sharp cannula 43 as described herein. Alternatively, the shaft 50 may be inserted without a cannula and the tip 150 may be guided through tension of the one or more suture legs 11. This configuration may be applied to any of the shafts 30, 50 described herein.

The tips 31, 150 described herein can be composed of any material suitable for piercing, cutting, and/or displacing tissue as required for a particular application. Such material could include stainless steel, surgical stainless steel, titanium, aluminum, brass, platinum, plastics such as polyetheretherketones (PEEK™) available from Victrex PLC of Lancashire, England, and/or polymers.

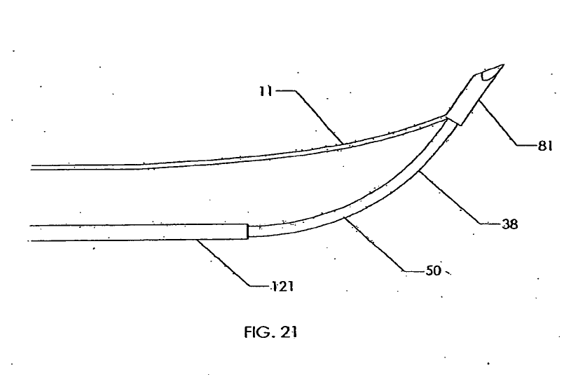

FIG. 21 depicts a flexible shaft 50 with a flexible spring form distal portion 38 of the shaft 50 is more flexible that the proximal portion 121 of the flexible shaft 50. Differential flexibility depicted in FIG. 21 may be achieved in several embodiments. In a first embodiment, distal portion 38 may be composed of a first material and proximal portion 121 may be composed of a second material wherein the first material is more flexible than the second material. The distal portion 38 and the proximal portion may be attached by any of several means known of those of skill in the art including, but not limited to, swaging, brazing, welding, soldering, threading, adhesives, epoxies, and/or fasteners. In a second embodiment, the distal portion 38 and the proximal portion 121 are composed of the same material, but the distal portion 38 and/or the proximal portion are heat treated to induce different physical properties such as flexibility. In a third embodiment, the first portion 38 has a different diameter than the second portion 121. This varying diameter may be achieving through a number of methods, including assembly of two separate pieces as described above, casting, rolling, and/or machining.

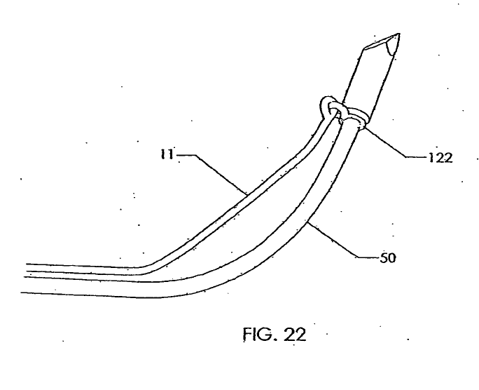

FIG. 22 depicts an embodiment of a flexible shaft element whereby the suture 11 is attached to the flexible shaft 50 by means of a wire loop eyelet suture retention means 122.

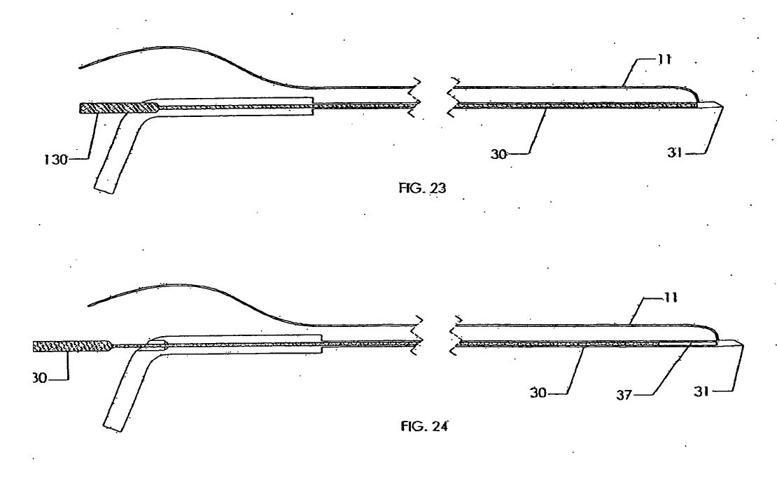

FIG. 23 and FIG. 24 depict a sequence of steps whereby an internal stiffening element 130 enables the flexible shaft 30 to be rigid during tissue penetration whereby removal of the stiffening element 130 effects lateral movement of the tip 31. FIG. 23 depicts an internal stiffening element 130 inserted into the flexible shaft lumen 37, making the flexible shaft 30 rigid during tissue penetration. FIG. 24 depicts the flexible shaft with the stiffening element withdrawn, effecting lateral movement of the sharp tip 31. The flexible shaft lumen may be formed by any methods known to those of skill in the art including but not limited to casting, machining, and rolling. While this embodiment does not require a stiff outer cannula to penetrate tissue, such a cannula could be added.

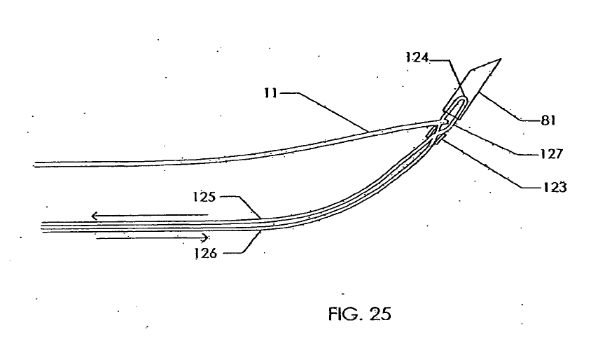

FIG. 25 depicts a bimorph embodiment of a flexible shaft portion of the surgical end effector whereby the flexible shaft comprises a wire loop with two legs of wire 125, 126 extending proximally. The wire loop forms a suture receiving eyelet 124 with a collar 123 near the tip. Two legs of the wire loop 125, 126 extend proximally. A tension leg 125 may be pulled to effect lateral motion of the wire loop tip 127. A second wire leg 126 can be held in place or pushed to further effect lateral motion of the wire loop tip. The wire loop tip 127 is depicted with an optional swaged tip 81 attached.

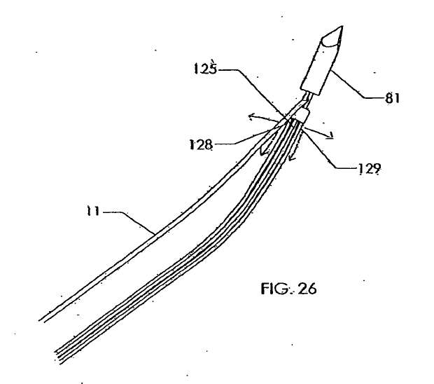

FIG. 26 depicts a flexible shaft whereby three or more push-pull elements 125, 128, 129 may be used to impart motion in additional lateral axes or to impart more force by gang action. A tension leg 125 imparts lateral motion in one lateral axis while left leg 128 and a right leg 129 impart lateral motion to the left and right.

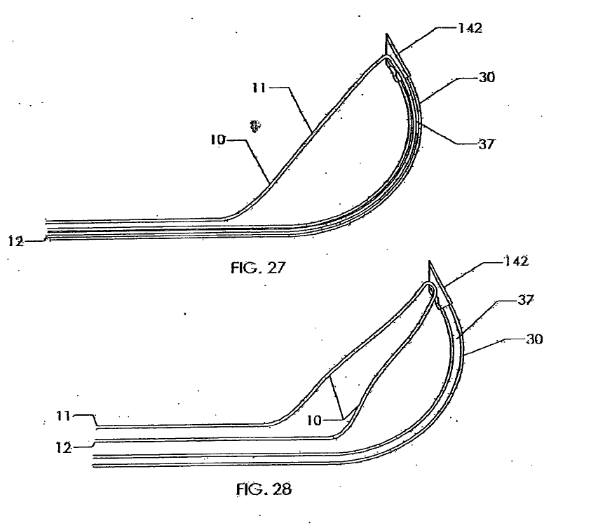

FIG. 27 and FIG. 28 depict an embodiment of a flexible shaft 30 with a lumen 37 whereby the flexible shaft 30 comprises a two-opening sharp tip head 142 whereby the two openings communicate with the lumen 37 of the shaft 30. FIG. 27 depicts one option of using this embodiment where one leg 12 of the suture 10 is run through the lumen 37 and the other leg 11 of the suture is external to the lumen 37. FIG. 28 depicts a second option of using this embodiment whereby both legs 11, 12 of the suture 10 are run outside the lumen 37 of the flexible shaft 30.

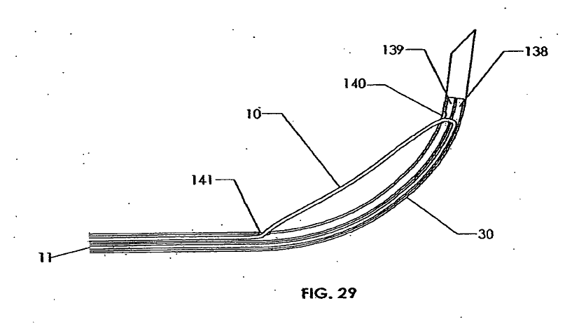

FIG. 29 depicts an embodiment of a flexible shaft having two lumens 138, 139. One leg 11 of suture 10 may be run through a first lumen 138 and exit the flexible shaft 30 through a distal hole 140 whereby the distal hole 140 communicates with the second lumen 139. The suture 10 may travel for a distance outside the flexible shaft 30 and then reenter the shaft 30 through a more proximal hole 141 that communicates with a second lumen 139 of the flexible shaft 30.

The surgical end effector can also be used for injecting adhesives, blood derived materials such as platelets, living cells, growth factors, insulin, or other substances into lesions or graft interfaces. This can be performed after the step of delivering suture, but before the suture is finally tied, or it can be used for non-suture repairs to apply adhesives, or agents to difficult recesses in the body where a small laterally displacing delivery means is desired. An example would be to inject liquid therapeutic agents to the prostate by transurethral puncture.

Fluids may be injected through the sharp cannula 43 and/or one or more lumens in a hollow flexible shaft 30. To enable fluid injection, an interface may be added to the hollow flexible shaft for receiving pressuring fluid. The sharp cannula 43 may include a fluid injector such as a nozzle to inject fluid into the cannula. In order to achieve desired pressure and directional flow of the liquid, a gasket (not shown) may be added to the sharp cannula 43 to prevent pressure loss from the proximal end of the sharp cannula 43 while allowing movement of the flexible shaft 30, 50. The gasket may be composed of materials such as rubber, elastomers, or other materials familiar to those of skill in the art. The ergonomic handle 90 may comprise a trigger or button for injecting fluid. Additionally or alternatively, fluid injection may be triggered by foot actuation, voice actuation, computer actuation, and/or a robotic surgical system.

As used herein, fluid is given a broad definition to include any flowable substances. For example, gases may be injected using the surgical end effector. In other embodiments, aerosolized solids or plastic solids may be injected using the surgical end effector.

FIG. 30 depicts a use of the surgical end effector to inject a flowable material into a region of tissue. In FIG. 30, a specific example of injecting a liquid therapeutic agent into a prostate gland is depicted. A prostate gland 68 is accessed by first passing a hollow flexible shaft 30 into the urethra 67 with the shaft 30 withdrawn into the sharp cannula 43 as depicted in FIG. 18. The sharp tip 31 of the flexible shaft is advanced to the tissue treatment region 69. A liquid therapeutic agent is passed through the flexible shaft lumen 37 by means of a flexible shaft liquid injection means 35 (refer to FIG. 17). Injectable therapeutic agents can comprise any drug or agent having a therapeutic and/or palliative effect on the tissue treatment region 69. For example, for prostate treatments, therapeutic agents can include, but are not limited to solutions containing alpha-blockers such as terazosin, prazosin, and doxazosin that relax the muscles of the prostate, agents that cause the prostate to shrink such as finasteride and dutasteride, and anti-tumor drugs such as cisplatin.

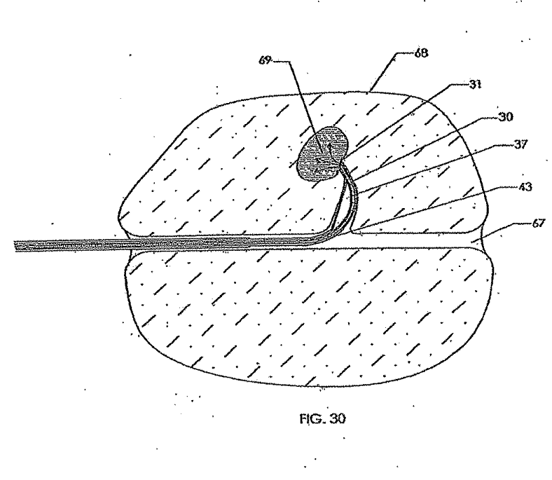

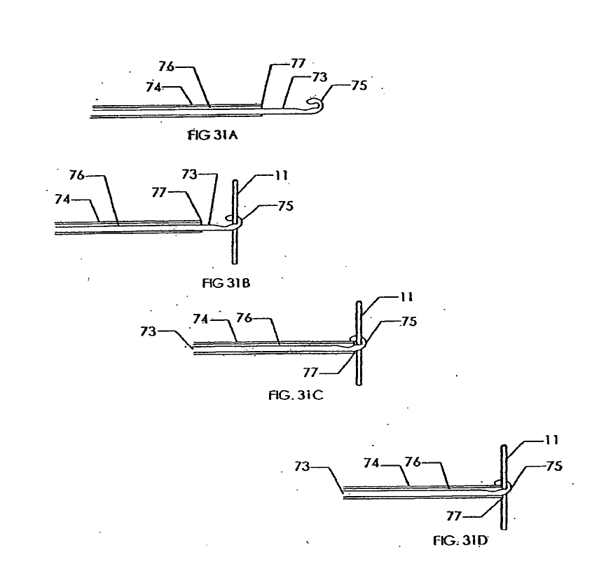

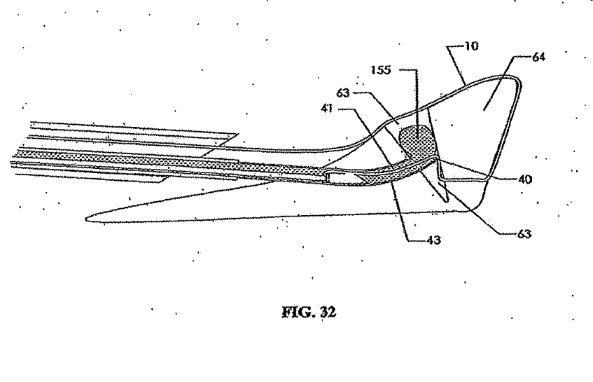

FIG. 31A depicts a slidable/sheathed suture retrieval means which consists of an inner retriever 73 with suture retrieval hook 75 slidably positioned in the lumen 76 of an outer sheath 74 with a distal end 77. FIG. 31B, FIG. 31C, and FIG. 31D depict a method of using the slidable/sheathed suture retrieval means to apprehend a suture leg 11. In FIG. 31B, the inner retriever 73 is extended so that the retrieval hook 75 extends past the distal end 77 of the outer sheath 74 and apprehends a suture leg 11. FIG. 31C depicts a further step whereby the inner retriever 73 has been withdrawn into the lumen 76 of the outer sheath 74 so that the suture leg 11 is trapped between the inner surface of the hook 75 and the distal end of the outer sheath 74. The suture leg 11 is thus apprehended within the confines of the inner surface of the hook 75 but can still slide through the hook 75 opening. FIG. 31D depicts a further step whereby the inner retriever 73 has been withdrawn further into the lumen 76 of the outer sheath 74 so that the suture leg 11 is wedged between the hook 75 and the distal end 77 of the outer sheath 74, thereby preventing the suture leg 11 from sliding within the confines of the hook 75.

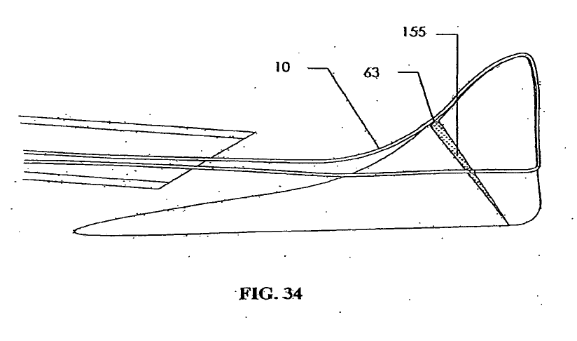

FIG. 32 depicts flowable adhesive being applied through the sharp cannula lumen 41. This step follows deployment of a suture leg 13 as depicted in FIG. 14 and occurs prior to full retraction of the sharp cannula 43 as depicted in FIG. 15. The sharp cannula 43 is withdrawn until the sharp cannula tip 40 is within the tissue tear 63. Flowable adhesive 155 is injected into the tissue tear 63 through the sharp cannula lumen 41. This step is followed by withdrawal of the sharp cannula 43 from the meniscus and tensioning of the suture 10 as depicted in FIG. 34. Tissues adhesives are well known to those of skill in the art and may include cyanocrylates such 2-octyl cyanoacrylate, n-butyl-cyanoacrylate, and/or fibrin glues. Examples of suitable tissue adhesives are described in U.S. Pat. No. 6,582,713 to Newell et al., which is incorporated herein by reference.

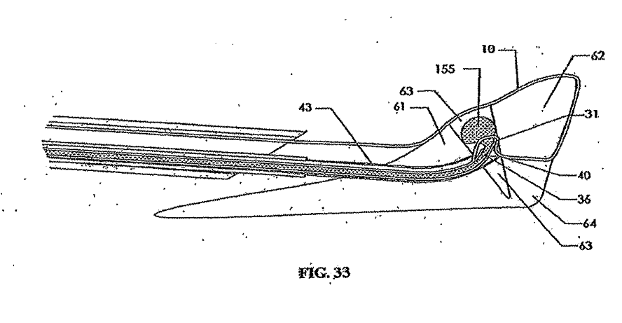

FIG. 33 depicts flowable adhesive 155 being applied through the flexible shaft lumen 36. This step would follow deployment of a suture leg 13 using a hollow lumen flexible shaft 30 in a manner analogous to that depicted in FIG. 14 and would occur prior to full retraction of the sharp cannula 43 as depicted in FIG. 15. The sharp cannula 43 is withdrawn until the sharp cannula tip 40 is within the tissue tear 63. The flexible shaft is advanced from the sharp cannula until the flexible shaft tip 31 is located at the desired adhesive placement region. Flowable adhesive 155 is injected into the tissue tear 63. This step is followed by rapid retraction of the flexible shaft 30 back into the sharp cannula and subsequent withdrawal of the sharp cannula 43 from the meniscus 61, 62, 64 and tensioning of the suture as depicted in FIG. 34.

FIG. 34 depicts the step following either FIG. 32 or 33. The sharp cannula 43 has been removed from the meniscus. Tension is applied to the suture 10 to close the tissue tear 63, compressing the adhesive 155 within the tissue tear 63 gap.

Injection of therapeutic agents as described above is not limited to medicines or adhesives. Rather, therapeutic agents can include blood constituents, cells, growth factors, nutrients, growth scaffolds, insulin, naked plasmid DNA, bioactive agents, and elements. Bioactive agents include, but are not limited to, silver, gold, platinum, zinc and alloys thereof.

In one embodiment, the flexible shaft 30, 50 is pre-bent and utilizes spring rebound when the shaft tip 31 extends beyond the inserter tube. In another embodiment, the flexible shaft 30, 50 is not pre-bent and utilizes a tension element to control the bending motion after the shaft 30, 50 exits the cannula lumen 41. In an embodiment, the flexible shaft 30, 50 utilizes a curved opening or a lateral deflector means in the cannula 43 opening to induce lateral travel of the flexible shaft tip when it is advanced 31.

The flexible shaft 30, 50 may be composed of a variety of materials. For example, the flexible shaft 30, 50 may be composed shape memory alloys such as nickel-titanium (nitinol), copper-zinc-aluminum, and copper-aluminum-nickel. (Nitinol is an acronym for Nickel Titanium Ordnance Laboratories.) The flexible shaft may be composed of spring steel, e.g. steel alloys containing silicon. The flexible shaft 30, 50 may additionally or alternatively be composed of a ferromagnetic shape memory alloy (FSMA) which changes shape under strong magnetic fields. In other embodiments, the flexible shaft 30, 50 may be composed of materials that change shape in response to changes in temperature, e.g. insertion into the body. The wire legs 125, 126 and push pull elements 128, 129 may be composed of the same or similar materials.

The flexible shaft 30, 50 need not have a memory property as tension from suture 10 may be sufficient in many cases to cause desired lateral and/or distal displacement. In some embodiments, the flexible shaft 30, 50 may comprise polymers. In some embodiments, a coating may be applied the solid flexible shaft 50, for example, a non-stick coating.

An embodiment comprises a hollow flexible shaft made with coiled wire rope. The wire rope construction enables the suture to be placed through the side of the flexible shaft by drawing the suture between the filars of wire to enable a releasable gripping of suture without damaging it. Suitable coiled wire rope is available from Asahi Intecc Co., Ltd. of Aichi, Japan.

Numerous means to form a coacting stop or travel limiting structure at the operator interface including, but not limited to, suture clamps, cleats, friction slots, capstans, and similar structures known to those skilled in the art are within the spirit and the scope of the presently disclosed embodiments. A means to lock and release the sliding and rotation of the flexible shaft with respect to the insertion cannula may also be used in the surgical end effector.

The invention described herein includes automated means of suturing. For example, referring again to FIG. 1, the ergonomic handle 90 may further comprise a trigger, button, or other actuating device. When the actuating device is pressed, the shaft 50 extends beyond the sharp cannula 43, stretching an end 11 of the suture 10 to form a bowstring array. The suture retrieval means 70 extends to retrieve the suture 11. In some embodiments, the surgical end effector may fastener the suture 10. Means of fastening may include, but are not limited to tying the suture 10, fusing the suture 10 through heat or chemical means, crimping a piece of metal or plastic over the suture 10, and joining the suture 10 with an adhesive or an epoxy.

While a surgeon or other medical or veterinary personnel may use the invention described herein, the invention is by no means limited to such uses. Rather, the invention may be used by a robot for surgery. Such embodiments have great potential with the rise of telemedicine as a surgeon could manipulate a robot from a local or remote location.

To facilitate remote surgery or enhance local surgery, the cannula may be configured to receive one or more sensors. For example, the cannula could receive a small video camera, e.g. a needle scope. In another embodiment, the cannula could receive a position sensor.

All patents, patent applications, and published references cited herein are hereby incorporated by reference in their entirety. It will be appreciated that various of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

1. A surgical end effector comprising:
an elongated cannula having a sharp distal end and a proximal end engaging an operator interface;
a shaft assembly slidably disposed within the cannula, the shaft assembly comprising a tip that moves from a first laterally confined position to a second position when the tip extends beyond the distal end of the cannula;
a suture receiver on the tip for engaging a suture; and
a suture apprehension retriever to releasably capture a laterally disposed suture to tie or otherwise connect with another portion of the same suture.

2. The surgical end effector of claim 1, wherein the shaft assembly is pre-bent.

3. The surgical end effector of claim 1, wherein the elongated cannula comprises a curved distal portion.

4. The surgical end effector of claim 1 further comprising a suture tension element to laterally displace the tip.

5.-8. (canceled)

9. The surgical end effector of claim 1 wherein the shaft assembly comprises two lumens and two holes, wherein a suture is releasably conveyed through a first lumen, passes out of the shaft assembly through a first hole, passes into the shaft assembly through a second hole and is releasably conveyed through a second lumen.

10.-11. (canceled)

12. The surgical end effector of claim 1 wherein the operator interface applies tension to a suture.

13. (canceled)

14. The surgical end effector of claim 1 wherein the suture apprehension retriever comprises:
an outer sheath having a lumen and a distal end; and
an inner retriever having a distal retrieval hook, wherein the inner retriever is slidably positioned in the lumen of the outer sheath.

15. A surgical end effector comprising:
an elongated shaft assembly having a distal end and a proximal end;
a flexible portion of the shaft assembly located toward the distal end of the shaft assembly;
a tip located at the distal end of the shaft assembly, the tip being movable from a first position to a second flexed position; and
a suture receiver located adjacent to the tip.

16. The surgical end effector of claim 15 further comprising a suture engaging the suture receiver.

17. The surgical end effector of claim 15 further comprising a suture apprehension retriever to releasably capture a laterally disposed suture to tie with another portion of the same suture.

18. The surgical end effector of claim 15 further comprising an elongated tube having a sharp distal end and a proximal end engaging an operator interface.

19. The surgical end effector of claim 18 wherein the elongated shaft assembly is slidably and rotationally movable within the elongated tube.

20. The surgical end effector of claim 15 further comprising a tension element adjacent to the tip.

21. The surgical end effector of claim 15, wherein the tip is a cutting tip.

22. The surgical end effector of claim 15, wherein the tip is detachable.

23. (canceled)

24. The surgical end effector of claim 15, wherein the shaft assembly comprises a plurality of legs wherein tip movement is achieved by pushing a first subset of the legs while pulling a second subset of the legs.

25. A method for surgical suture placement comprising:
penetrating tissue with an elongated substantially straight sharp instrument;
advancing an elongated shaft within the sharp instrument, the shaft comprising a tip extending from a flexible portion, the tip movable in at least one lateral direction and a suture engaged to a suture holder located on the tip of the shaft; and
applying tension to a suture tension element or other tensioning means to move the tip and suture holder to a lateral position.

26. The method of claim 25 further comprising:
rotating the suture holder and suture tension element.

27. The method of claim 25 further comprising:
retrieving the suture with a releasable slidable retriever.

28. A surgical end effector comprising:
a flexible shaft, the shaft having a sharp distal tip and a internal lumen;
a suture receiver located toward the distal tip; and
an internal stiffening element received in the lumen that enables the flexible shaft to be rigid during tissue penetration, wherein the stiffening element may be withdrawn from the flexible shaft to effect lateral movement of the tip while managing suture tension.

29. The flexible shaft of claim 28 wherein the internal stiffening element is a wire.

30. The flexible shaft of claim 28 wherein a suture forms a taught bowstring array.

31. (canceled)

32. The flexible shaft of claim 28 wherein a proximal end of the internal stiffening element is partially withdrawn from the flexible shaft while a distal end of the internal stiffening element remains in the flexible shaft.

33. A surgical end effector for fluid injection comprising:
a shaft assembly comprising:
a shaft;
a tip located at the distal end of the shaft, the tip being movable from a first position to a second flexed position;
a fitting interface; and
a lumen in communication with the fitting interface, wherein fluid can be received through the fitting interface and the lumen before exiting the lumen.

34.-38. (canceled)

39. The surgical end effector of claim 33, wherein the shaft assembly further comprises a tension element adjacent to the tip.

40.-41. (canceled)

42. A The surgical end effector of claim 1, further comprising:
a fluid injector for injecting fluids through the elongated cannula.

43.-44. (canceled)
